(19) United States
(12) Patent Application Publication
(10) Pub. No.: US 2005/0234418 A1
(43) Pub. Date: Oct. 20, 2005

(54) Sanitary napkin

(75) Inventors: Wataru Yoshimasa, Kagawa (JP); Yuuki Noda, Kagawa (JP); Megumi Tokumoto, Mitoyo-gun (JP); Noritatsu Tamagawa, Kagawa (JP)

Correspondence Address: DARBY & DARBY P.C., P. O. BOX 5257, NEW YORK, NY, 10150-5257, US

(73) Assignee: Uni-Charm Corporation, Shikokuchuo-shi (JP)

(21) Appl. No.: 11/097,522

(22) Filed: Apr. 1, 2005

(30) Foreign Application Priority Data
Apr. 15, 2004 (JP) 2004-119855

(51) Int. Cl. A61F 13/15; A61F 13/20

(52) U.S. Cl. 604/378; 604/358

(57) ABSTRACT

Disclosed is a sanitary napkin which is allowed to quickly absorb menstrual blood and also effective in preventing absorbed menstrual blood from returning to the skin-side surface under pressure and in concealing the color of absorbed menstrual blood from the outside. Between a liquid absorbent layer and a topsheet formed of a resin film having a large number of liquid passage apertures, there is disposed a large number of barrier members having a small width. The barrier members are strips formed of a resin film, and gaps are left between adjacent barrier members. After passing through the liquid passage apertures of the topsheet, menstrual blood tends to be absorbed by the liquid absorbent layer through the gaps. The barrier members also function to prevent liquid return and conceal the color of menstrual blood.

### BACKGROUND OF THE INVENTION

1. Field of the Invention

The present invention relates to a sanitary napkin which is effective in preventing lateral leakage and concealing the color of menstrual blood absorbed in a liquid absorbent layer. More particularly, the invention relates to a sanitary napkin which is intended to diffuse an applied liquid longitudinally beneath a topsheet for quick absorption into the liquid absorbent layer.

2. Description of the Related Art

Sanitary napkins are typically constructed to include a liquid-permeable topsheet, a backsheet, and a liquid absorbent layer (or absorbent core) disposed between the topsheet and the backsheet. The sanitary napkins need to be able to absorb menstrual blood in a short period of time and to be effective in preventing lateral leakage of an applied liquid and also in preventing menstrual blood absorbed in the liquid absorbent layer from returning to the skin-side surface of the topsheet, and it is also desired that the color of menstrual blood absorbed in the liquid absorbent layer is less noticeable from the outside. To satisfy these requirements, there have been developed topsheets of various structures and additional components to be disposed between the topsheet and the liquid absorbent layer.

Patent Publication 1 identified below discloses a sanitary napkin which is intended to facilitate intake of liquid, to be effective in preventing liquid return to the skin-side surface of the topsheet, and to improve the function of concealing the color of liquid. In the sanitary napkin disclosed in Patent Publication 1, more specifically, a first passage layer (topsheet) located on its skin-side surface is a resin film formed with large openings having an individual open area of 1.4 to 3.0 mm² and a second passage layer located beneath the first passage layer is formed of highly-porous lofty elastic fibrous material. Also disclosed is a structure to be compared with the invention, where the same first passage layer is employed but the second passage layer is replaced by a resin film formed with small openings.

Patent Publication 2 identified below discloses a sanitary napkin in which an aggregate of crimped fibers is disposed between the topsheet and the liquid absorbent layer. These crimped fibers are entangled together to have a given elastic modulus. Due to the presence of the aggregate of crimped fibers disposed between the topsheet and the liquid absorbent layer, the sanitary napkin can easily conform to the vaginal opening.

In FIGS. 11 and 12 of Patent Publication 3 identified below, on the other hand, there is disclosed a sanitary napkin having a pair of skin-contacting parts that is elongated in a longitudinal direction of the sanitary napkin and raised toward the wearer's skin. In each skin-contacting part, a bundle of continuous filaments opened from a tow is disposed between the topsheet and the liquid absorbent layer with their fiber extending direction brought into line with the longitudinal direction.

In this invention, the pair of skin-contacting parts intended to contact the woman's crotch has cushioning properties. In addition, since menstrual blood discharged from the vaginal opening can be received in the groove defined between the skin-contacting parts for subsequent absorption by the liquid absorbent layer, menstrual blood is inhibited from flowing laterally on the skin-side surface of the sanitary napkin, thereby preventing lateral leakage of menstrual blood.

Patent Publication 1: Japanese Unexamined Patent Publication No. H11-507573

Patent Publication 2: Japanese Unexamined Patent Publication No. 2001-340382

Patent Publication 3: Japanese Unexamined Patent Publication No. 2002-345887

In the invention disclosed in Patent Publication 1, liquid-permeability of the first passage layer is improved by the large openings, and the second passage layer formed of the fibrous material is disposed between the first passage layer and the liquid absorbent layer. When body pressure is exerted on the skin-side surface, therefore, menstrual blood absorbed in the liquid absorbent layer is liable to pass through the second passage layer to return to the skin-side surface of the first passage layer through the large openings. In addition, since menstrual blood is allowed to be retained in the voids of the second passage layer, the color of menstrual blood is noticeable from the outside through the large openings of the first passage layer.

In the comparative structure disclosed in Patent Publication 1, on the other hand, the resin film having small openings is provided as the second passage layer beneath the first passage layer. This second passage layer appears to be able to make the color of menstrual blood inside the liquid absorbent layer less noticeable from the outside. However, the second passage layer also functions as a barrier against menstrual blood tending toward the liquid absorbent layer after passing through the first passage layer. Therefore, menstrual blood is liable to remain between the first passage layer and the second passage layer and this residual menstrual blood tends to diffuse along a plane between the first passage layer and the second passage layer. This easily leads to lateral leakage. In addition, the residual menstrual blood easily returns to the skin-side surface of the first passage layer through the large openings.

In the sanitary napkin disclosed in Patent Publication 2, on the other hand, the aggregate of crimped fibers facilitate deformation of the skin-side surface to conform to the body shape, but there is no particular mention of the function of facilitating penetration of menstrual blood into the liquid absorbent layer and the function of preventing liquid return from the liquid absorbent layer to the skin-side surface. The aggregate of crimped fibers disposed beneath the topsheet functions such that an agglutinate of menstrual blood with relatively high viscosity is cut by the crimped fibers after passing through the topsheet to thereby cause worry that it becomes difficult for menstrual blood to migrate toward the liquid absorbent layer.

In the sanitary napkin disclosed in Patent Publication 3, the pair of skin-contacting parts is provided for leading menstrual blood along the groove defined therebetween. However, the individual skin-contacting parts are not designed to be highly effective in transferring menstrual blood into the liquid absorbent layer.

### SUMMARY OF THE INVENTION

The present invention has been developed to solve the problems in the prior art set forth above and has an object to provide a sanitary napkin which is allowed to quickly intake menstrual blood with relatively high viscosity and is effective in preventing lateral diffusion and concealing the color of menstrual blood.

According to the invention, there is provided a sanitary napkin comprising
a liquid-permeable topsheet intended to face the vaginal opening of a wearer,
a backsheet located on opposite side of the topsheet, and
a liquid absorbent layer located between the topsheet and the backsheet, the sanitary napkin further comprising
a plurality of barrier members located between the topsheet and the liquid absorbent layer, the individual barrier members being elongated longitudinally of the sanitary napkin and having a flattened cross-section with one major surface directed toward the topsheet and made of synthetic resin,
wherein the barrier members are stacked such that gaps are left between the barrier members to communicate with the liquid absorbent layer. Preferably, the barrier members remain unbonded to each other.

In the sanitary napkin of the present invention, the barrier members beneath the topsheet function to prevent liquid return from the liquid absorbent layer to the topsheet and to conceal the color of menstrual blood absorbed in the liquid absorbent layer. Due to the gaps defined between the longitudinally extending barrier members, moreover, menstrual blood after passing through the topsheet can be diffused longitudinally along the gaps for subsequent absorption by the liquid absorbent layer. Accordingly, menstrual blood can be quickly transferred to the liquid absorbent layer. This also results in prevention of lateral diffusion of menstrual blood so as to be effective in preventing lateral leakage of menstrual blood.

Furthermore, since at least the skin-side surface of each barrier member is made of resin, the barrier members can exhibit an elastic restoring force when they are deformed by a body pressure. Accordingly, the gaps can be maintained to allow passage of menstrual blood.

Preferably, some of the barrier members are bonded to the topsheet. With this construction, even if a body pressure is exerted on the sanitary napkin, the barrier members are less apt to cause displacement beneath the topsheet. Accordingly, the gaps are appropriately distributed in the stack of the barrier members to effectively maintain permeability to menstrual blood.

Preferably, the barrier members are serpentine or crimped so that the gaps can be appropriately distributed in the stack of the barrier members.

Preferably, the flattened cross-section of the barrier member has a width of 0.1 to 5 mm and a thickness of 0.02 to 3.0 mm.

Preferably, the topsheet has a skin-side surface made of resin and a plurality of liquid passage apertures, and the flattened cross-section of the barrier member has a width smaller than an opening size of the liquid passage apertures as measured laterally of the sanitary napkin. In this construction, since the liquid passage apertures are not completely closed by the barrier members, menstrual blood is allowed to readily transfer from the topsheet to the liquid absorbent layer.

Preferably, the barrier members are whitened so as to further improve the effect of making the color of menstrual blood absorbed in the liquid absorbent layer less noticeable from the outside.

### BRIEF DESCRIPTION OF THE DRAWINGS

FIG. 1 is a plan view showing a sanitary napkin according to a first embodiment of the present invention;

FIG. 2 is a sectional view of the sanitary napkin of FIG. 1 taken along line II-II;

FIG. 3 is a partial perspective view schematically showing how a topsheet and barrier members are disposed on a liquid absorbent layer;

FIG. 4 is a partial enlarged view of FIG. 2;

FIG. 5 is an enlarged sectional view showing a modification of the sanitary napkin of FIG. 1;

FIG. 6 is an enlarged sectional view showing another modification of the sanitary napkin of FIG. 1;

FIG. 7 is an enlarged perspective view showing another embodiment of the barrier members;

FIGS. 8(A) and 8(B) are enlarged perspective views showing other embodiments of the barrier members;

FIG. 9 is a view schematically showing a process for manufacturing the barrier members; and

FIG. 10 is a plan view showing a sanitary napkin having a different structure according to the present invention.

### DESCRIPTION OF THE PREFERRED EMBODIMENT

It should be noted that the sanitary napkin, as well as its individual components, has two major surfaces: of which one surface intended to be worn toward the wearer's crotch is referred to as “skin-side surface”, while the other surface is referred to as “garment-side surface”.

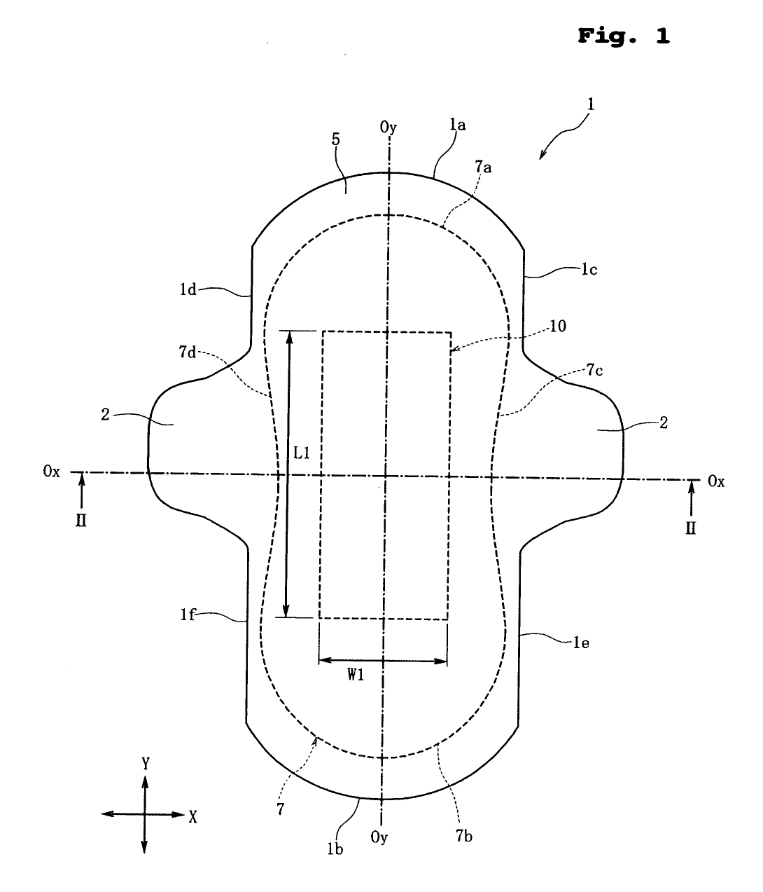

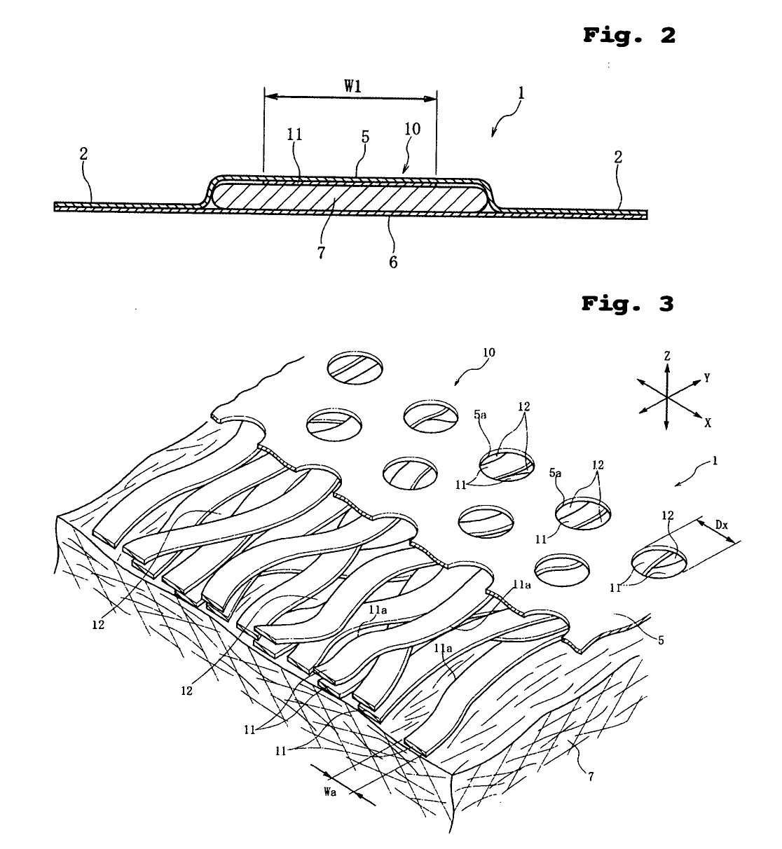

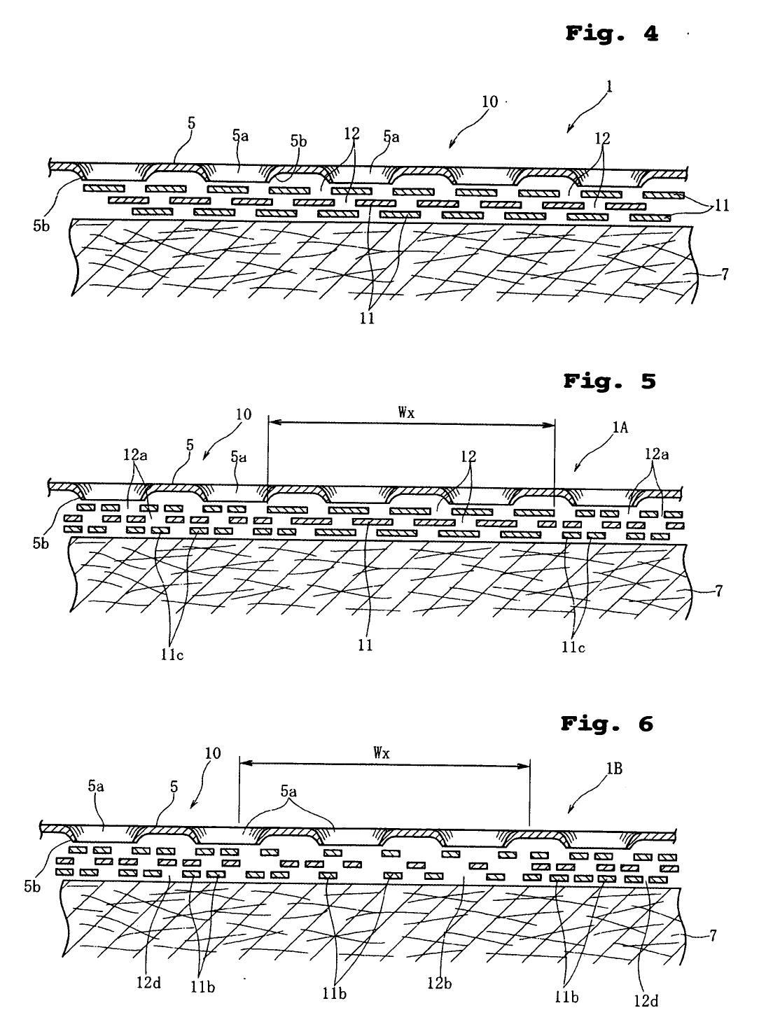

FIG. 1 is a plan view of a sanitary napkin 1 according to a first embodiment of the present invention with its skin-side surface directed to this side; FIG. 2 is a sectional view taken along line II-II of FIG. 1; FIG. 3 is a partial perspective view schematically showing how a topsheet and barrier members are disposed on a liquid absorbent layer; and FIG. 4 is a partial enlarged view of FIG. 2.

As shown in FIG. 1, the sanitary napkin 1 has a main body elongated in a Y-direction (or longitudinal direction) and bilaterally symmetrical about a longitudinal centerline Oy. The main body has front and rear edges 1a, 1b that are bowed (or bulged) forward and rearward, respectively. From laterally opposite sides of the main body, wings 2, 2 project outward. These wings 2, 2 are not aligned with a lateral centerline Ox and are located closer to the front edge 1a than to the rear edge 1b. The main body has forward right and forward left edges 1c, 1d forward of the wings 2, 2 and rearward right and rearward left edges 1e, 1f rearward of the wings 2, 2.

As shown in FIG. 2, the sanitary napkin 1 is basically constructed of a liquid-permeable topsheet 5 appearing on the skin-side surface of the sanitary napkin 1, a liquid-blocking backsheet 6 appearing on the garment-side surface of the sanitary napkin 1, and a liquid absorbent layer 7 disposed between the topsheet 5 and the backsheet 6. As indicated by dotted line in FIG. 1, the liquid absorbent layer 7 has a front edge 7a that is bowed (or bulged) forward and spaced inward from the front edge 1a of the sanitary napkin 1 and a rear edge 7b that is bowed (or bulged) rearward and spaced inward from the rear edge 1b of the sanitary napkin 1. The liquid absorbent layer 7 also has a right edge 7c that is spaced inward from the forward right edge 1c and the rearward right edge 1e of the sanitary napkin 1 and a left edge 7d that is spaced inward from the forward left edge 1d and the rearward left edge 1f of the sanitary napkin 1. Outside the liquid absorbent layer 7, the topsheet 5 and the backsheet 6 are bonded together through a hot-melt type adhesive.

If desired, additional liquid-impermeable side sheets may be located a given distance apart from the longitudinal centerline Oy to cover laterally opposite side portions of the skin-side surface of the sanitary napkin 1. The side sheets may also be used to provide leakage preventing walls (or cuffs) which extend longitudinally and are raised away from the skin-side surface.

In the sanitary napkin 1, the region enclosed by dotted line is designated main liquid-receiving region 10. The main liquid-receiving region 10 is symmetrical about both the longitudinal centerline Oy and the lateral centerline Ox and has a length L1 and a width W1, as shown in FIG. 1. In the main liquid-receiving region 10, barrier members 11 are disposed between the topsheet 5 and the liquid absorbent layer 7.

The barrier members 11 are resin film strips having a width Wa and extending continuously over the length L1 of the main liquid-receiving region 10, as shown in FIG. 3. If desired, the individual barrier members 11 may be shorter than the length L1 of the main liquid-receiving region 10.

Between the topsheet 5 and the liquid absorbent layer 7, the barrier members 11 are stacked in a Z-direction (thickness direction) of the sanitary napkin 1 into a multilayered structure. Unless otherwise noted, the barrier members 11 thus stacked remain unbonded to each other.

Between the barrier members 11, there are left a large number of gaps 12. Since the individual barrier members 11 extend generally in the Y-direction, the individual gaps 12 are elongated in the Y-direction. The gaps 12 provide paths through which menstrual blood transfers from the topsheet 5 to the liquid absorbent layer 7. Here, some of the gaps 12 provide through-holes which pass through the stack of the barrier members 11 in the Z-direction to allow for direct transfer of menstrual blood from the topsheet 5 to the liquid absorbent layer 7. As shown in FIG. 3, more specifically, the individual barrier members 11 are not straight strips parallel to the Y-direction but serpentine strips with curved (or bowed) portions 11a randomly repeated in the Y-direction. The curved portions 11a contribute to increasing the porosity due to the gaps 12.

The width Wa of the individual barrier member 11 is in the range of 0.1 to 5.0 mm, preferably in the range of 0.5 to 3.0 mm. Within this range, the barrier members 11 are prevented from interfering with transfer of menstrual blood from the topsheet 5 to the liquid absorbent layer 7.

The thickness of the individual barrier members 11 is in the range of 0.02 to 3.0 mm, preferably in the range of 0.5 to 2.0 mm. When body pressure is exerted on the skin-side surface of the sanitary napkin 1, the barrier members 11 may sometimes be deformed to clear the gaps 12. If the thickness of the individual barrier members 11 is set within the above range, however, the barrier members 11 thus deformed can easily recover to their original state due to resilience when the body pressure is relieved.

The volume ratio of the gaps 12 to the space defined between the garment-side surface of the topsheet 5 and the skin-side surface of the liquid absorbent layer 7 in the main liquid-receiving region 10, i.e., the porosity of the stack of the barrier members 11 due to the gaps 12 is in the range of 20% to less than 100%. The stack of the barrier members 11 has a height in the range of 0.5 to 10 mm, preferably in the range of 2 to 7 mm.

If the height of the stack of the barrier members 11 is set within the above range, high viscosity menstrual blood having passed through the topsheet 5 can pass through the gaps 12 in an aggregated state to reach the liquid absorbent layer 7. If the porosity due to the gaps 12 is set within the above range, also, menstrual blood having passed through the topsheet can smoothly flow down the barrier members 11 to reach the liquid absorbent layer 7.

The length L1 and the width W1 of the main liquid-receiving region 10 are so set as to allow the main liquid-receiving region 10 to easily face the vaginal opening when the sanitary napkin 1 is worn in the crotch of a woman. The preferred range of the length L1 is (but not limited to) from 40 to 70 mm and the preferred range of the width W1 is (but not limited to) from 20 to 30 mm.

Suitable thermoplastic resin materials for the barrier members 11 includes, but not limited to, LLDPE (linear low-density polyethylene), LDPE (low-density polyethylene), HDPE (high-density polyethylene), PP (polypropylene), TAFMER® by Mitsui Chemicals, INC., and blends thereof. These resin materials may also be combined to form a multi-layered film. The resin film may be whitened by kneading the resin material with inorganic filler such as titanium oxide, barium sulfate or calcium carbonate in an amount of 10 to 40 wt. %. If the topsheet 5 and the barrier members 11 are whitened, the color of menstrual blood absorbed in the liquid absorbent layer 7 can be effectively concealed. The resin material used for the barrier members 11 may be apertured. Moreover, the resin material used for the barrier members 11 may be treated to be hydrophilic by adding a hydrophilizing agent such as esters in an amount of 0.1 to 2.5 wt. %.

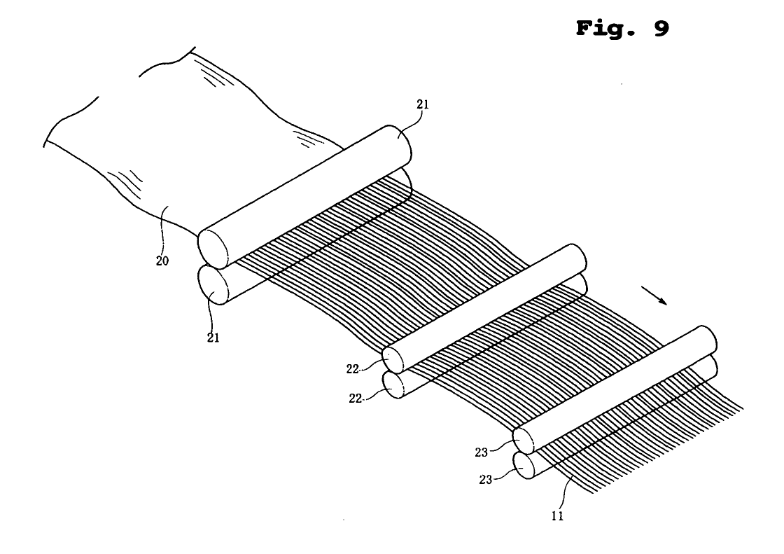

For example, the barrier members 11 may be manufactured according to a method shown in FIG. 9.

Film 20 having a given width is made of any one of the above-mentioned resin materials and fed in a single-ply or multi-ply arrangement between a pair of cutter rolls 21, 21 to be cut into a number of strips (barrier members 11). After cutting, the strips are let out while being held between nip rolls 22, 22 and nip rolls 23, 23. At this stage, if the peripheral speed of downstream nip rolls 23, 23 is faster than that of the upstream nip rolls 22, 22, the strips are given tension between the nip rolls 22, 22 and the nip rolls 23, 23 and then released from the tension after passing through the nip rolls 23, 23. By this process, the barrier members 11 can be separated from each other so as not to adhere to each other.

In order to make the individual barrier members 11 in the serpentine shape of FIG. 3, the cutter rolls 21, 21 may have serpentine cutting blades (not shown) on their roll surface. More specifically, the individual cutting blades may travel the entire circumference of the cutter rolls 21, 21 to have an edge winding as viewed in the direction of the normal to the roll surface. In an alternative, the individual barrier members 11 may be made in the serpentine shape such that the film is cut into straight strips by using the cutter rolls 21, 21 and then subjected to application and release of tension several times by using nip rolls.

The barrier members 11 may also be manufactured such that the synthetic resin film being stretched is torn in a direction perpendicular to the drawing direction, the resin film being stretched is torn by pressure of jets of water or hot air, or the resin film is fed between a pair of embossing rolls in meshing engagement with each other and torn by tension exerted by the meshing rolls.

In the sanitary napkin 1 according to the first embodiment, the topsheet 5 is formed of an apertured synthetic resin film, i.e., a synthetic resin film in which a large number of liquid passage apertures 5a are regularly or randomly distributed.

At least within the main liquid-receiving region 10 where the barrier members 11 are provided, the individual liquid passage apertures 5a have an opening diameter (or opening size) Dx which is measured in an X-direction (or lateral/transverse direction) of the sanitary napkin 1 and is preferably larger than the width Wa of the individual barrier members 11, as shown in FIG. 3. If the width Wa of the individual barrier members 11 is smaller than the opening diameter Dx, the gaps 12 are allowed to show through each liquid passage aperture 5a. In FIG. 3, the liquid passage apertures 5a are of a substantially round shape.

The liquid passage apertures 5a may be of an oval shape elongated in the X-direction. For example, a resin film having a given percent open area due to liquid passage apertures having a given individual open area may be stretched to increase both the percent open area and the individual open area and then placed on the barrier members 11 with the drawing direction coinciding with the X-direction. The topsheet 5 thus prepared is convenient for making the opening diameter Dx of the liquid passage apertures 5a larger than the width Wa of the barrier members 11.

If desired, the width Wa may be larger than the opening diameter Dx. Even in this case, it is still possible to transfer menstrual blood from the topsheet 5 to the liquid absorbent layer 7 as long as the barrier members 11 are treated to be hydrophilic.

The resin film used for the topsheet 5 is made of the same resin material as the barrier members 11 and whitened and treated to be hydrophilic in the same manner as the barrier members 11.

The opening diameter Dx of the individual liquid passage apertures 5a in the topsheet 5 is in the range of 0.3 to 6.0 mm, preferably in the range of 1.0 to 3.0 mm. The percent open area of the topsheet 5 due to the liquid passage apertures 5a is in the range of 10 to 60%, preferably in the range of 20 to 50%.

The liquid passage apertures 5a may be formed by a perforation method, for example, such that the resin film is softened on a substrate having a number of apertures and air is suck from the underside of the substrate. According to this perforation method, the individual liquid passage apertures 5a are tapered toward the barrier members 11 to have rings (or short tubes) 5b projecting from the garment-side surface of the resin film, as shown in FIG. 4.

Preferably, some of the barrier members 11 are bonded to the topsheet 5. For example, the topsheet 5 may be bonded to the stack of the barrier members 11 through a hot-melt type adhesive which is applied in an amount of 1 to 10 g/m² in a pattern of laterally extending strips or a spiral pattern so as not to interfere with liquid transfer.

In an alternative, the topsheet 5 may be fusion-bonded to the stack of the barrier members 11 by heating them under pressure to have embossments arranged in a dot pattern. Such heat-embossing also leads to partial bonding of barrier members 11 overlapping in the Z-direction, but sufficient gaps can still be left between the barrier members 11 such as by adopting a suitable embossing pattern. The embossments may occupy 1 to 40%, preferably 1 to 15% of the area (L1×W1) of the main liquid-receiving region 10. The individual embossments may have an area of 1.75 to 28.26 mm², preferably 7 to 19 mm².

The backsheet 6 is formed of a liquid-blocking sheet such as a PE (polyethylene) resin film. The liquid absorbent layer 7 is constructed by wrapping a mixture of fluff pulp and superabsorbent polymer (SAP) in tissue. So as not to interfere with liquid transfer, the liquid absorbent layer 7 is bonded to the stack of the barrier members 11 through a hot-melt type adhesive applied in an amount of 1 to 10 g/m². The backsheet 6 is also bonded to the liquid absorbent layer 7 through a hot-melt type adhesive.

When wearing, the sanitary napkin 1 is placed on the inner side of a crotch part of an undergarment and fixed to the crotch part through a pressure-sensitive adhesive layer provided on the garment-side surface (backsheet 6) of the main body of the sanitary napkin 1. Then, the wings 2, 2 are folded back and adhered to the outer side of the crotch part of the undergarment through pressure-sensitive adhesive layers provided on the garment-side surface (backsheet 6) of the wings 2, 2.

When the sanitary napkin 1 is put on the crotch of a wearer, the main liquid-receiving region 10 faces the vaginal opening so that menstrual blood discharged from the vaginal opening can be applied mainly to the main liquid-receiving region 10. Since the gaps 12 are allowed to show through each liquid passage aperture 5a, menstrual blood can easily transfer from the skin-side surface of the topsheet 5 to the liquid absorbent layer 7.

In addition, since the gaps 12 are elongated in the Y-direction, the gaps 12 facilitates diffusion of menstrual blood in the Y-direction. Thus, menstrual blood is inhibited from migrating in the X-direction to thereby prevent lateral leakage from the sanitary napkin 1.

On the other hand, since the barrier members 11 are stacked in a multilayered structure beneath the liquid passage apertures 5a of the topsheet 5, as shown in FIG. 3, menstrual blood absorbed in the liquid absorbent layer 7 is effectively prevented from returning to the skin-side surface of the topsheet 5. In addition, the liquid passage apertures 5a tapered toward the barrier members 11 to have the rings 5b are resistant to back-flow of menstrual blood. This also contributes to preventing menstrual blood from returning to the skin-side surface of the topsheet 5.

Since the topsheet 5 and the barrier members 11 are whitened and the barrier members 11 are located beneath the liquid passage apertures 5a of the topsheet 5, as set forth above, the color of menstrual blood absorbed in the liquid absorbent layer 7 is less noticeable from the outside.

FIGS. 5 and 6 are enlarged sectional views showing modifications of the sanitary napkin according to the first embodiment.

FIG. 5 shows a sanitary napkin 1A in which the barrier members 11 having the same width Wa as those used in the sanitary napkin 1 according to the first embodiment are disposed in a center region Wx (i.e., an area having a width Wx in the X-direction with center at the longitudinal centerline Oy). Laterally outside the center region Wx, on the other hand, there are disposed narrower barrier members 11c having a width in the range of ¼ to ¾ of the width Wa of the barrier members 11. The unit weight of the barrier members 11 disposed in the center region Wx may be equal to or slightly smaller than that of the barrier members 11c outside the center region Wx. Accordingly, individual gaps 12a outside the center region Wx tend to be smaller than the individual gaps 12 in the center region Wx.

FIG. 6 shows a sanitary napkin 1B in which barrier members 11b are disposed between the topsheet 5 and the liquid absorbent layer 7. The barrier members 11b have an identical width but are disposed such that the unit weight of the barrier members 11b disposed outside the center region Wx is larger than that of the barrier members 11b in the center region Wx. Accordingly, the volume ratio of gaps 12b to the space defined between the garment-side surface of the topsheet 5 and the skin-side surface of the liquid absorbent layer 7 in the center region Wx is larger than the volume ratio of gaps 12d to the space defined between the garment-side surface of the topsheet 5 and the skin-side surface of the liquid absorbent layer 7 outside the center region Wx.

In the sanitary napkin 1A of FIG. 5 and the sanitary napkin 1B of FIG. 6, menstrual blood can reach the liquid absorbent layer 7 faster in the center region Wx. On the other hand, prevention of return of menstrual blood from the liquid absorbent layer 7 to the topsheet 5 is more effective outside the center region Wx. The width Wx is in the range of 10 to 50 mm, preferably in the range of 15 to 35 mm.

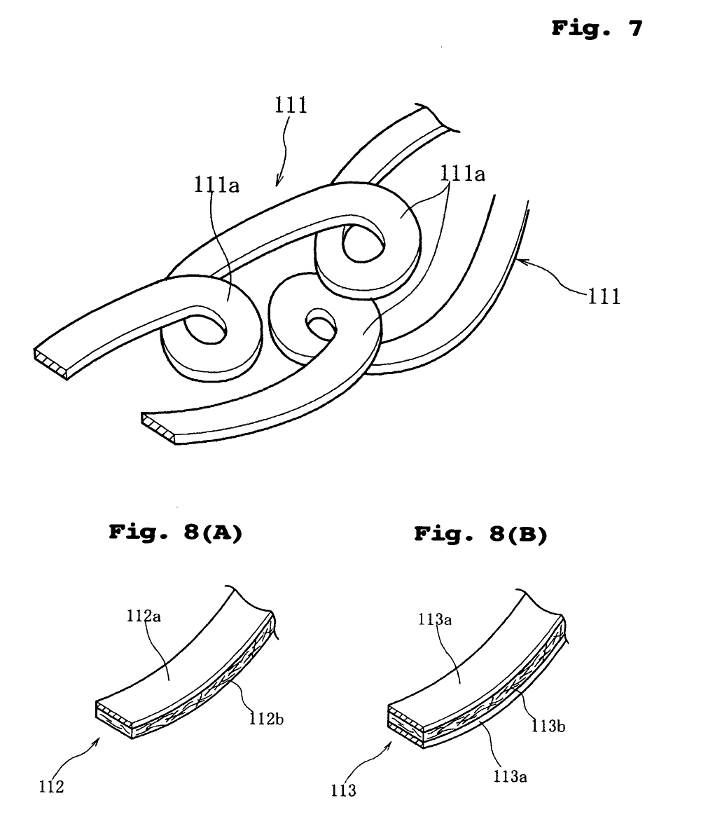

FIGS. 7 and 8 show other embodiments of the barrier members.

FIG. 7 shows barrier members 111 which are made of the same resin material as the barrier member 11 but are crimped to have loops 111a. The barrier members 111 thus crimped easily leave gaps in the stack thereof.

FIG. 8(A) shows a barrier member 112 having a resin film layer 112a whitened and treated to be hydrophilic in the same manner as the barrier member 11 of the foregoing embodiments and a fibrous layer 112b laid on and joined to the resin film layer 112a. The barrier members 112 are disposed with the resin film layer 112a directed toward the topsheet 5. FIG. 8(B) shows a barrier member 113 in which a fibrous layer 113b similar to the fibrous layer 112b is sandwiched between and joined to resin film layers 113a, 113a similar to the resin film layer 112a.

The fibrous layers 112b, 113b are hydrophilic nonwoven fabrics joined to the resin film layers 112a, 113a such as by adhesive-bonding or fusion-bonding. For example, the fibrous layers 112b, 113b may be fusion-bonded to the resin film layers 112a, 113a to have embossments in a dot pattern. Suitable fibers for the fibrous layers 112b, 113b are synthetic resin fibers treated to be hydrophilic, such as PE, PP or PET (polyethylene terephthalate). The fibrous layers 112b, 113b may further contain cellulosic fibers. Since the hydrophilic fibrous layers 112b, 113b appear on the side edges of the barrier members 112, 113, as shown in FIGS. 8(A) and 8(B), menstrual blood after passing through the liquid passage apertures 5a of the topsheet 5 can be strongly drawn to the gaps defined between adjacent barrier members.

It should be noted that various barrier members according to the foregoing embodiments may be mixed with each other or distributed in different locations.

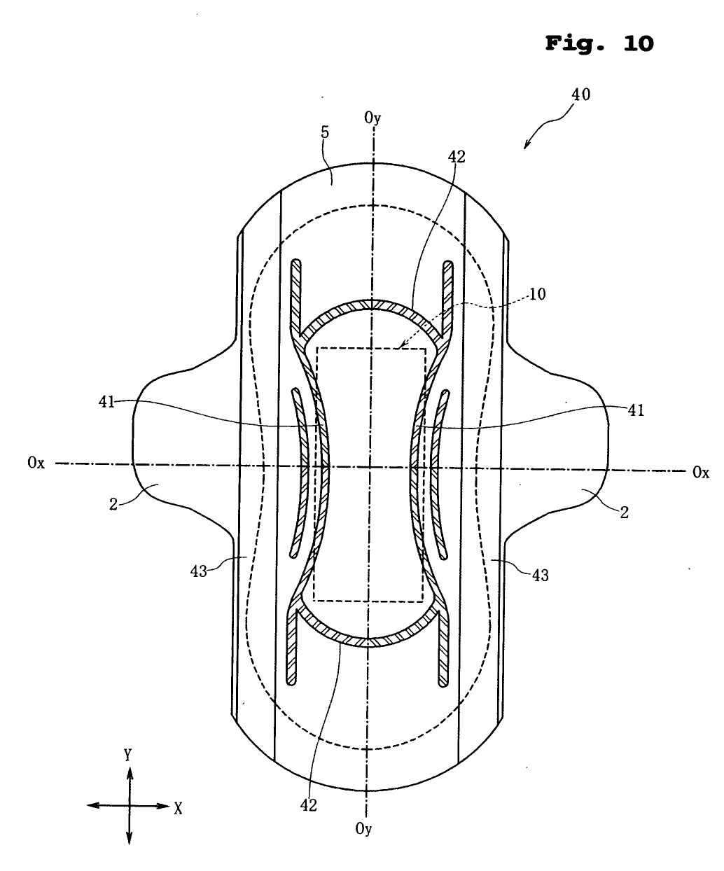

FIG. 10 is a plan view of a sanitary napkin 40 having a different structure according to the present invention. The sanitary napkin 40 further includes: longitudinal compressed grooves 41, 41 overlapping with laterally opposite side portions of the main liquid-receiving region 10 where the barrier members 11 are present; and lateral compressed grooves 42, 42 spaced forward and rearward from the main liquid-receiving region 10.

Although the present invention has been illustrated and described with respect to exemplary embodiments thereof, it should be understood by those skilled in the art that the foregoing and various other changes, omission and additions may be made therein and thereto, without departing from the spirit and scope of the present invention. Therefore, the present invention should not be understood as limited to the specific embodiments set out above but to include all possible embodiments which can be embodied within a scope encompassed and equivalent thereof with respect to the feature set out in the appended claims.

1. A sanitary napkin comprising:
a liquid-permeable topsheet for facing the vaginal opening of a wearer;
a backsheet located on the opposite side of the topsheet;
a liquid absorbent layer located between the topsheet and the backsheet;
a plurality of barrier members located between the topsheet and the liquid absorbent layer, the individual barrier members being elongated longitudinally of the sanitary napkin and having a flattened cross-section with one major surface directed toward the topsheet and made of synthetic resin;
wherein the barrier members are stacked such that gaps are left between the barrier members to communicate with the liquid absorbent layer.

2. A sanitary napkin according to claim 1, wherein the barrier members remain unbonded to each other.

3. A sanitary napkin according to claim 1, wherein some of the barrier members are bonded to the topsheet.

4. A sanitary napkin according to claim 1, wherein the barrier members are serpentine or crimped.

5. A sanitary napkin according to claim 1, wherein the flattened cross-section of the barrier member has a width of 0.1 to 5 mm and a thickness of 0.02 to 3.0 mm.

6. A sanitary napkin according to claim 1, wherein the topsheet has a skin-side surface made of resin and a plurality of liquid passage apertures, and the flattened cross-section of the barrier member has a width smaller than an opening size of the liquid passage apertures as measured laterally of the sanitary napkin.

7. A sanitary napkin according to claim 1, wherein the barrier members are whitened.
